(19) United States
(12) Patent Application Publication
(10) Pub. No.: US 2026/0207685 A1
(43) Pub. Date: Jul. 23, 2026

(54) LACTIPLANTIBACILLUS PLANTARUM GOLDGUT-HNU082 AND USE THEREOF IN ALLEVIATING DEPRESSION

(71) Applicant: SHENZHEN PORSHEALTH BIOENGINEERING CO., LTD., SHENZHEN (CN)

(72) Inventors: SILU ZHANG, SHENZHEN (CN); JUN WANG, SHENZHEN (CN); JIE PAN, SHENZHEN (CN); TING SHU, SHENZHEN (CN); GUOXUN XIAO, SHENZHEN (CN)

(21) Appl. No.: 19/328,601

(22) Filed: Sep. 15, 2025

(30) Foreign Application Priority Data
Jan. 20, 2025 (CN) 202510082793.4

(51) Int. Cl. A61K 35/747; A61P 25/24; C12N 1/20; C12R 1/25

(52) U.S. Cl. CPC A61K 35/747; A61P 25/24; C12N 1/205; C12R 2001/25

(57) ABSTRACT

Lactiplantibacillus plantarum GOLDGUT-HNU082 and a use of the Lactiplantibacillus plantarum GOLDGUT-HNU082 in alleviating depression are provided. This Lactiplantibacillus plantarum GOLDGUT-HNU082 is deposited in the China General Microbiological Culture Collection Center, with the deposit number of CGMCC No. 33075 and deposit date of Dec. 16, 2024, and is taxonomically named Lactiplantibacillus plantarum.

### CROSS REFERENCE TO RELATED APPLICATIONS

The present disclosure claims the priority to the Chinese patent application with the filing No. 202510082793.4, entitled “Lactiplantibacillus plantarum GOLDGUT-HNU082 AND USE THEREOF” and filed on Jan. 20, 2025 with the Chinese Patent Office, the contents of which are incorporated herein by reference in their entirety.

### TECHNICAL FIELD

The present disclosure relates to the technical field of microorganisms, and more particularly to Lactiplantibacillus plantarum GOLDGUT-HNU082 and use thereof in alleviating depression.

### BACKGROUND ART

Depression is a common and serious mental disorder that affects more than 280 million people worldwide. Depression has become one of the leading causes of physical disability, and severe cases may even lead to suicide. Although existing antidepressant treatment methods such as psychotherapy and drug therapy have alleviated patients' symptoms to a certain extent, there are still problems such as limited efficacy and significant side effects. Antidepressant medications with good efficacy, such as agomelatine, escitalopram, vortioxetine and fluoxetine, still have high treatment failure rates, and side effects including nausea, rash, liver and kidney damage, etc. Therefore, it is particularly important to explore new, safe and effective strategies for the prevention and treatment of depression.

In recent years, an increasing number of researches have demonstrated the crucial role of the gut-brain axis (GBA) in regulating mood and behavior. The gut-brain axis refers to the interaction between the gut microbiome, the intestinal tract, the immune system, and the central nervous system. The gut microbiome plays a crucial role in brain function and mood regulation by influencing the nervous system, immune system, endocrine system, and metabolic pathway. Probiotics, as a means of regulating the gut microbiome, can regulate and control the gut microbiome to produce various neurotransmitters and metabolites, such as serotonin, γ-aminobutyric acid, dopamine, and norepinephrine. These substances enter the brain via the pneumogastric nerves or the enteric nervous system, to influence the mood and behavior.

Hence, the problem that needs to be urgently solved by those skilled in the art is how to alleviate depression by regulating the gut-brain axis through probiotics.

### SUMMARY

In view of this, an object of the present disclosure is to provide Lactiplantibacillus plantarum GOLDGUT-HNU082 and its use in alleviating depression to address the deficiencies in the prior art.

In order to achieve the above object, the present disclosure adopts the following technical solutions:
Lactiplantibacillus plantarum GOLDGUT-HNU082, which strain is deposited in the China General Microbiological Culture Collection Center (CGMCC), with the deposit address of Institute of Microbiology, Chinese Academy of Sciences, NO. 3, Courtyard 1, Beichen West Road, Chaoyang District, Beijing, the deposit number of CGMCC NO. 33075, and the deposit date of Dec. 16, 2024, and has the taxonomic name of Lactiplantibacillus plantarum.

The Lactiplantibacillus plantarum GOLDGUT-HNU082 of the present disclosure is isolated from fermented food. Whole genome sequencing results show that GOLDGUT-HNU082 has great potential for development as a probiotic in terms of physiological functions.

The present disclosure aims to systematically evaluate the potential role of GOLDGUT-HNU082 in the prevention and treatment of depression by inducing depression in mice with chronic unpredictable mild stress. A series of behavioral tests are performed to assess depressive-like behaviors in mice, and to measure inflammatory cytokines and neurotransmitter levels in the brain and serum. Furthermore, the mouse gut microbiota (gut flora) and metabolites thereof are analyzed by using a fecal shotgun metagenomic technology to comprehensively explore the effects of GOLDGUT-HNU082 on mouse physiology and gut microbiota, providing new insights into the antidepressant application of the probiotic GOLDGUT-HNU082.

The present disclosure further seeks for protection for a use of the Lactiplantibacillus plantarum GOLDGUT-HNU082 in preparation of a medicine for alleviating depression.

Further, the above depression is caused by unpredictable mild stress (CUMS).

It can be seen from the above technical solutions that compared with the prior art, the beneficial effects of the present disclosure are as follows.
1. The Lactiplantibacillus plantarum GOLDGUT-HNU082 of the present disclosure can significantly alleviate depressive-like behaviors in mice caused by chronic unpredictable mild stress, including reducing anxiety and despair behaviors and increasing pleasure, thereby alleviating depression; significantly increase the levels of key neurotransmitters such as serotonin (5-HT), dopamine (DA), γ-aminobutyric acid (GABA) and norepinephrine (NE) in the colon, serum and brain, and regulate and control emotional responses; significantly reduce the levels of interleukin 6 (IL-6), tumor necrosis factor α (TNF-α) and lipopolysaccharide (LPS), to alleviate inflammatory responses; reduce brain neuronal damage; and improve the gut microbiota dysbiosis in depressed mice, to reduce harmful bacteria and increase the abundance of beneficial bacteria.
2. Metagenomic results further show that the present disclosure elucidates the intrinsic mechanism of GOLDGUT-HNU082 in chronic unpredictable mild stress-induced depression, providing a solid scientific basis for the development of probiotic-based microbial products as an alternative treatment strategy for alleviating depression.

### BRIEF DESCRIPTION OF DRAWINGS

FIG. 1 shows morphology image of GOLDGUT-HNU082;

FIG. 2 shows the whole genome comparison results of GOLDGUT-HNU082;

FIG. 3 shows the effects of GOLDGUT-HNU082 on depressive-like behaviors in mice;

FIG. 4 shows the effects of GOLDGUT-HNU082 on levels of key neurotransmitters in the colon, serum, and brain of mice;

FIG. 5 shows the effects of GOLDGUT-HNU082 on inflammatory responses in mice;

FIG. 6 shows the effects of GOLDGUT-HNU082 on mouse brain neurons; and

FIG. 7 shows the effects of GOLDGUT-HNU082 on the gut microbiota of mice

### DETAILED DESCRIPTION OF EMBODIMENTS

The technical solutions of embodiments in the present disclosure are clearly and completely described below. Apparently, the described embodiments are only some of the embodiments of the present disclosure, not all of the embodiments. Based on the embodiments of the present disclosure, all other embodiments obtained by those ordinarily skilled in the art without paying creative efforts are within the scope of protection of the present disclosure.

### Example 1

Isolation, Screening and Identification of Lactiplantibacillus plantarum GOLDGUT-HNU082

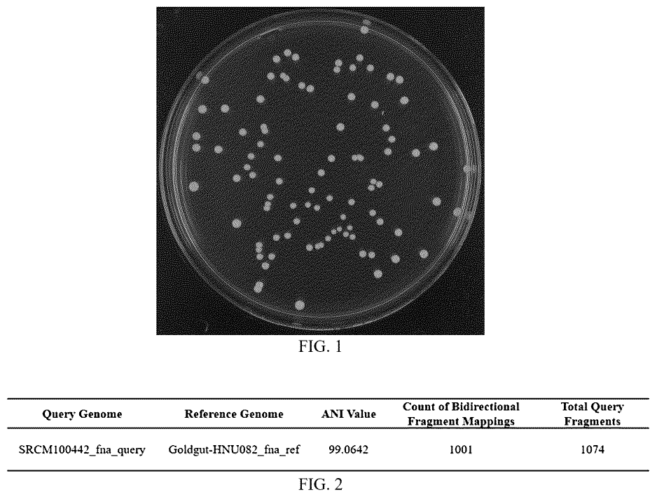

30 fermented food samples were collected from farmers'markets in various counties and cities in Hainan Province, including Wanning, Wenchang, Wuzhishan, Baisha, and Lingshui, etc. The collected fermented food samples were diluted tenfold in sterilized physiological saline, and subsequently prepared into sample dilutions at gradients of: 10⁻¹, 10⁻², 10⁻³, 10⁻⁴, and 10⁻⁵ by using a ten-fold dilution method. 0.1 mL of 10⁻³, 10⁻⁴, and 10⁻⁵ sample dilutions (sample dilutions at the gradients of 10⁻³, 10⁻⁴, and 10⁻⁵) were respectively spread onto a MRS agar medium, cultured anaerobically at 37° C. for 48 h, and subjected to multiple times of isolation, streaking, purification, culturing, and identification, to screen out 5 Lactiplantibacillus plantarum strains. The 5 strains were cultured in a MRS liquid for 24 h, and the OD value and viable cell count were measured, and finally, a Lactiplantibacillus plantarum strain with relatively good growth activity was screened out and named GOLDGUT-HNU082. The purified GOLDGUT-HNU082 strain was inoculated into a MRS broth medium and activated for 24 h, and then spread onto a MRS agar medium, and after culturing at 37° C. for 24 h, the bacterial colony was white, had a diameter of 0.1-0.2 cm, and had a smooth, moist, raised surface and a neat edge (as shown in FIG. 1). When observed under an optical microscope, the bacteria were rod-shaped.

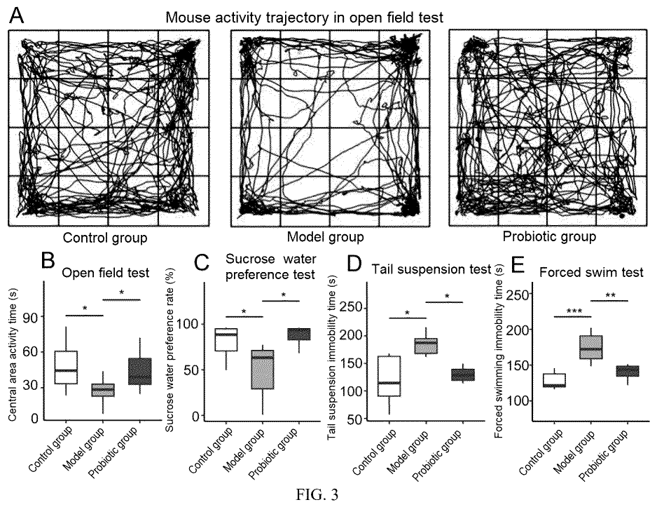

Identification Information: The GOLDGUT-HNU082 strain was subjected to whole genome sequencing. Sequences obtained from the whole genome sequencing were subjected to genome alignment. Genomic similarity analysis was performed using the online analysis platform Galaxy (https://usegalaxy.org/) and FastANI software (https://github.com/ParBLiSS/FastANI). The analysis results showed that the ANI value of the whole-genome sequence of GOLDGUT-HNU082 and that of Lactiplantibacillus plantarum SRCM100442 was 99.0642 (as shown in FIG. 2).

### Example 2

### 1 Experimental Materials

### 1.1 Materials and Animals

As in Example 1, GOLDGUT-HNU082 was isolated from fermented food.

24 5-week-old male C57BL/6J mice were purchased from Beijing Vital River Laboratory Animal Technology Co., Ltd. The common feed used in the experiment was obtained from Jiangsu Xietong Pharmaceutical Bio-Engineering Co., Ltd., China. The energy sources of the common feed were protein (22.9%), fat (11.1%), and carbohydrates (66%).

### 1.2 Experimental Design

During the experiment, the mice were housed in an SPF-grade animal laboratory with a 12-h light-dark cycle maintained, a temperature controlled at 23±2° C., and a humidity maintained at 55%±5%. The mice had free access to water and feed. After a week of acclimatization, the mice were randomly divided into three groups, with 8 mice in each group: a control group, a model group, and a probiotic group, respectively. Except for the control group, the mice were subjected to various repeated unpredictable stressors twice daily for eight weeks. The stressors included wet bedding, no bedding, restraint, cage tilt, altered light-dark cycle, flashing lights, and 4° C. cold water bath. On average, two kinds of stressors were applied daily. During this period, the mice in the control group and the model group were gavaged with physiological saline, while the mice in the probiotic group were gavaged with 1×10⁹ cfu of GOLDGUT-HNU082. After eight weeks, all mice were subjected to a series of behavioral tests. After the behavioral tests, fresh fecal samples of the mice were collected for metagenomic sequencing. All mice were fasted for 12 h, and then sacrificed by dislocation after the orbital blood was collected. The blood samples were left to stand at room temperature for 2 h, and then centrifuged at 3,000 rpm, 4° C. for 20 min to separate the serum, which was then aliquoted and stored at −80° C. for subsequent experiment. The colons and brains of the mice were also collected, a part of them were fixed in 4% paraformaldehyde solution, and the other part were rapidly frozen in liquid nitrogen and then stored at −80° C.

### 1.3 Physiological and Biochemical Index Evaluation

The levels of serotonin, dopamine, γ-aminobutyric acid, norepinephrine, interleukin-6, tumor necrosis factor α, and lipopolysaccharide in the serum, colon, and brain were measured using ELISA kits provided by Shanghai Xin Yu Biotech Co., Ltd.

### 1.4 Histopathological Analysis

The hippocampus was fixed in a 4% paraformaldehyde solution, dehydrated, embedded in paraffin, and then sectioned. After deparaffinization, antigen retrieval, and nonspecific binding blocking, the sections were incubated overnight respectively with primary antibodies against brain-derived neurotrophic factor (BDNF), primary antibodies against postsynaptic density-95 (PSD-95), and primary antibodies against glial fibrillary acidic protein (GFAP) which were labeled with a red fluorophore or a green fluorophore. Subsequently, the sections were reacted with fluorescently labeled secondary antibodies, subjected to nuclear staining with DAPI, and finally mounted with an antifade reagent for observation under a fluorescence microscope.

### 1.5 Fecal Metagenomic Sequencing and Microbial Species Annotation

The total DNA was extracted from mouse fecal sample using the QIAamp DNA Fecal Mini Kit (Qiagen Company, Hilden, Germany) according to the manufacturer's instructions, and the purity and integrity of the obtained DNA were assessed. Sequencing libraries were prepared using the Illumina NEBNext@Ultra DNA Library Preparation Kit (NEB, USA). DNA fragments of approximately 350 bp were obtained by ultrasonication and then subjected to shotgun metagenomic sequencing on an Illumina NovaSeq 2500 platform of Novogene Company (Beijing, China). Metagenomic data were analyzed using HUMAnN3 software to identify microbial species and predict metabolic pathway annotation and abundance information based on the MetaCyc database.

### 1.6 Animal Behavior Experiment

### 1.6.1 Sucrose Water Preference Test

The sucrose water preference test consists of two parts: an acclimation training part and a testing part respectively. During the training phase, two bottles of 1% (w/v) sucrose water solution were placed on each mouse cage. After 24 h, one of the bottles was replaced with a bottle of pure water. After 12 h, the water bottles were swapped, and the training ended 12 h later. Subsequently, the mice were deprived of food and water for 24 h. After 24 h, a bottle of 1% (w/v) sucrose water solution and a bottle of pure water were placed on the mouse cage for a 12-h sucrose water preference test. Sucrose water solution consumption and pure water consumption were recorded 12 h later. During the test, the mice were deprived of food. The sucrose water preference rate =sucrose water solution consumption/(sucrose water solution consumption+pure water consumption).

### 1.6.2 Open Field Test

Mice were placed in a constant-temperature constant-humidity environment with good sound-insulation effect for 1 h to acclimate. After 1 h, the mice were placed in the center of the bottom of the open field chamber. A video camera was used to record the mice's activities for the next 8 minutes, and the activity behaviors of the mice were analyzed using VisuTrack animal behavior software. Before each open field test, the chamber must be sterilized and deodorized with 75% medical alcohol first.

### 1.6.3 Tail Suspension Test

Mice were each suspended for 6 min with an adhesive tape at a position where it cannot escape, with 2 min for acclimation and 4 min for calculation of immobility time. In the above, the first 2 minutes were used for acclimation, and the last 4 minutes were used for calculation of immobility time. The shorter time for escape attempts (longer immobility time) during the last 4 minutes indicated a higher level of depression.

### 1.6.4 Forced Swim Test

Water (23-25° C.) was added into a plastic cylinder (with a diameter of 19 cm and a depth of 25 cm) to a depth of approximately 18 cm. The mice were placed individually in the water for 5 min. The animals'behaviors were recorded with a camera, and the immobility time was automatically assessed using software. Longer immobility time indicated a higher level of depression in the mice.

### 1.7 Statistical Analysis

Statistical analysis and data visualization were performed using GraphPad Prism and R software. Experimental values were expressed as mean ±standard error of the mean (SEM). Data significance was calculated using one-way analysis of variance, followed by Tukey's test for pairwise comparison. When data did not meet normal distribution or homogeneity of variance, Kruskal-Wallis test was used for analysis of significant difference, and post hoc pairwise comparisons were performed using Dunn's test. P<0.05 was considered statistically significant, p<0.01 was considered strongly statistically significant, and p<0.001 was considered very strongly statistically significant.

### 2 Results

### 2.1 GOLDGUT-HNU082 Alleviating Depressive-like Behaviors in Mice

Long-term chronic unpredictable mild stress induces depressive-like behaviors in mice, including decreased exploratory behavior, and increased anhedonia and despair behaviors. As shown in FIG. 3, the central area activity time (27.29 s) and sucrose water preference rate (48.56%) of mice in the model group are significantly lower than the central area activity time (49.15 s) and sucrose water preference rate (81.27%) of those in the control group; and the tail suspension immobility time (177.41 s) and forced swimming immobility time (174.41 s) of mice in the model group are significantly higher than the tail suspension immobility time (119.59 s) and forced swimming immobility time (123.24 s) of those in the control group. In contrast, the central area activity time (44.85 s) and sucrose water preference rate (85.39%) of GOLDGUT-HNU082-intervened mice in the probiotic group are significantly higher than those of the model group, while the tail suspension immobility time (132.23 s) and forced swimming immobility time (140.57 s) are significantly lower than those of the model group. In summary, GOLDGUT-HNU082 can significantly alleviate depressive-like behaviors in mice induced by chronic unpredictable mild stress.

### 2.2 GOLDGUT-HNU082 Regulating Levels of Key Neurotransmitters in the Colon, Serum, and Brain

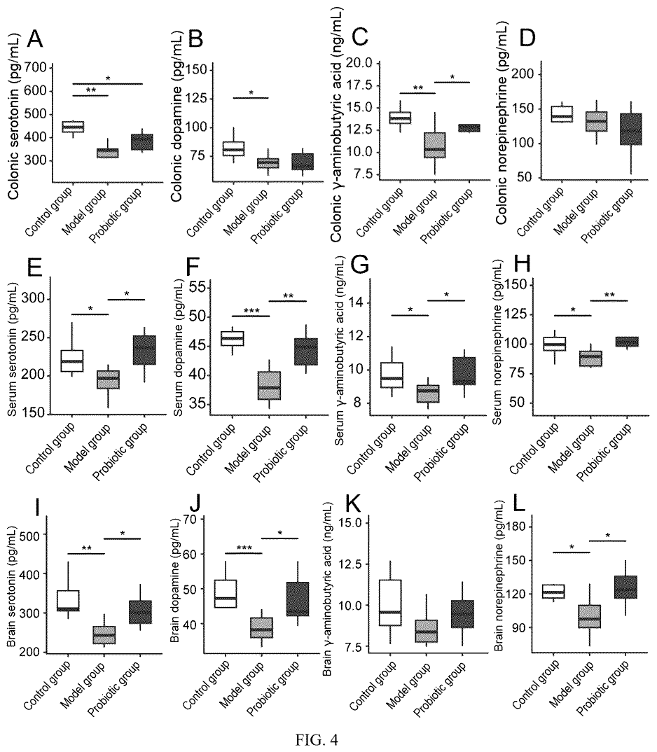

“Monoamine Hypothesis” is one of the leading hypotheses for the pathogenesis of depression. Decreased levels of monoamine neurotransmitters such as serotonin or norepinephrine have long been considered a main predisposing cause or physiological indicator. Depressed patients exhibit dysregulated levels of neurotransmitters such as serotonin, dopamine, γ-aminobutyric acid, and norepinephrine in the body. Therefore, we measured levels of related neurotransmitters in the colon tissues, serum, and brain tissues of mice. The results are as shown in FIG. 4. The levels of serotonin (330.49 pg/mL), dopamine (69.90 nmol/L), and γ-aminobutyric acid (10.60 ng/mL) in the colon of the mice in the model group are significantly lower than the levels of serotonin (446.39 pg/mL), dopamine (82.33nmol/L), and γ-aminobutyric acid (13.90 ng/mL) in the colon of the mice in the control group. The levels of serotonin (198.46 pg/mL), dopamine (38.20 nmol/L), γ-aminobutyric acid (8.62 ng/mL), and norepinephrine (84.44 pg/mL) in the serum of the mice in the model group are significantly lower than the levels of serotonin (225.65 pg/mL), dopamine (45.83 nmol/L), γ-aminobutyric acid (9.92 ng/mL), and norepinephrine (99.16 pg/mL) in the serum of the mice in the control group. The levels of serotonin (253.41 pg/mL), γ-aminobutyric acid (8.60 ng/mL), and norepinephrine (99.72 pg/mL) in the brain of the mice in the model group are significantly lower than the levels of serotonin (333.32 pg/mL), γ-aminobutyric acid (10.03 ng/mL), and norepinephrine (124.08 pg/mL) in the brain of the mice in the control group.

In contrast, the levels of γ-aminobutyric acid (12.64 ng/mL) in the colon, the serotonin (232.10 pg /L), dopamine (44.41 nmol/L), γ-aminobutyric acid (9.77 ng/mL), and norepinephrine (102.65 pg/mL) in the serum, and the serotonin (305.59 pg/mL), γ-aminobutyric acid (9.41 ng/mL), and norepinephrine (125.55 pg/mL) in the brain of the GOLDGUT-HNU082-intervened mice in the probiotic group are all significantly higher than those of the control group. The above results indicate that GOLDGUT-HNU082 can regulate levels of key neurotransmitters such as serotonin, and alleviate depression, providing new insights into the development of antidepressant measures.

### 2.3 GOLDGUT-HNU082 Attenuating Inflammatory Responses

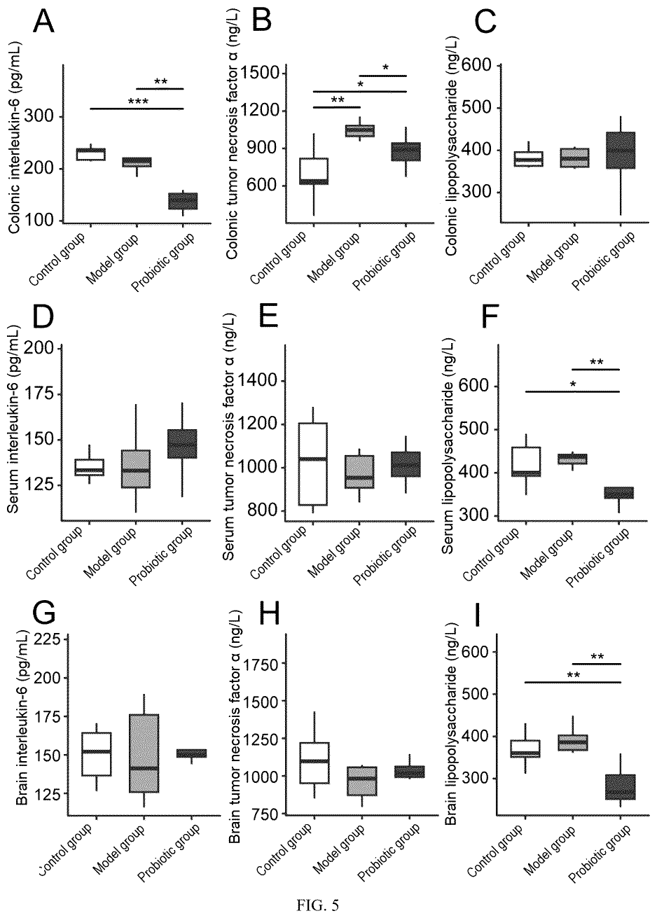

“Neuroinflammation” is also considered to be one of the predisposing causes of the pathogenesis of depression. To this end, we measured the levels of interleukin-6, tumor necrosis factor α and lipopolysaccharide in the colon tissues, serum and brain tissues of mice. The results are as shown in FIG. 5. The levels of tumor necrosis factor α (1018.68 ng/L) in the colon, lipopolysaccharide (446.14 ng/L) in serum, and lipopolysaccharide (381.25 ng/L) in the brain of the mice in the model group are significantly higher than the levels of tumor necrosis factor α (690.38 ng/L) in the colon, lipopolysaccharide (419.22 ng/L) in serum, and lipopolysaccharide (379.69 ng/L) in the brain of the control group, and the levels of tumor necrosis factor α (876.61 ng/L) in the colon, lipopolysaccharide (361.16 ng/L) in serum, and lipopolysaccharide (280.36 ng/L) in the brain of the mice in the probiotic group are significantly lower than those in the model group. Furthermore, there was no significant difference between the levels of interleukin-6 (214.75 pg/mL) in the colon of the model group and the interleukin-6 (225.05 pg/mL) in the colon of the control group. However, the level of interleukin-6 (144.27 pg/mL) in the colon of the probiotic group is significantly lower than those of the model group and the control group. The above results indicate that GOLDGUT-HNU082 can attenuate inflammatory responses in depression, which may help prevent the progression of depression disease.

### 2.4 GOLDGUT-HNU082 Alleviating Brain Neuronal Damage

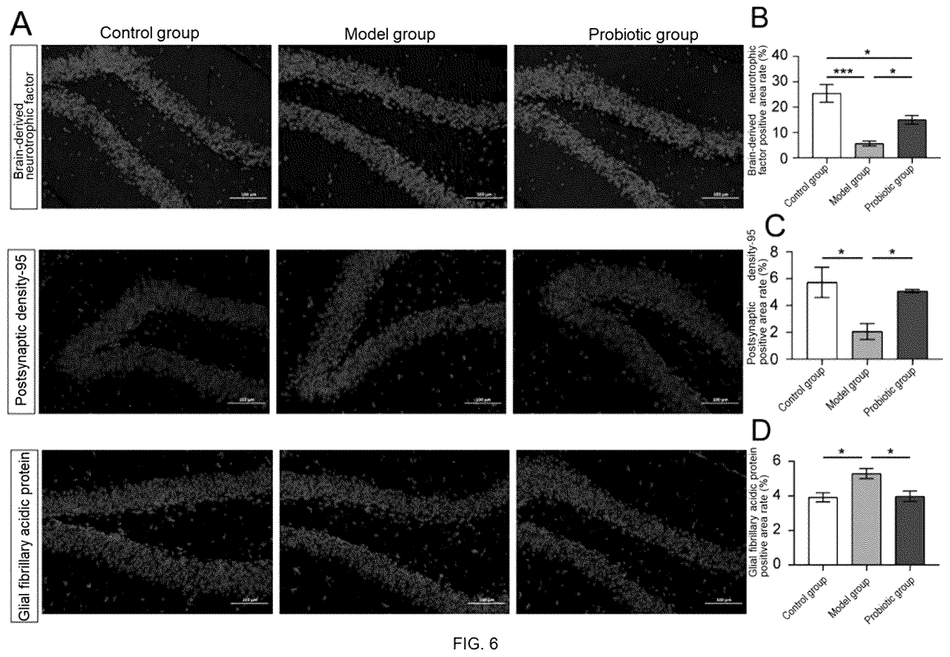

Due to dysregulation of neurotransmitter levels and occurrence of neuroinflammation, neuronal damage is often concomitant in the brains of patients with depression. In the above, brain-derived neurotrophic factor (BDNF) which is a key mediator of neuronal synaptic plasticity in the brain can promote neuronal growth and also the formation and stabilization of neuronal synapses in the brain. Postsynaptic density-95 (PSD-95) is a scaffolding protein that plays a key role in synaptic plasticity. Glial fibrillary acidic protein (GFAP) is a marker of astrocyte activation during neuronal damage. To further investigate the therapeutic effect of GOLDGUT-HNU082 on depression, we performed immunofluorescence staining on the DG region of the hippocampus of mice to observe neuronal damage situation. As shown in FIG. 6, compared with the control group, the BDNF and PSD-95 positive area ratios of the model group are significantly reduced, while the GFAP positive area ratio is significantly increased (A in FIG. 6, B in FIG. 6, C in FIG. 6, D in FIG. 6). After intervention by GOLDGUT-HNU082, compared with the Mod group, the BDNF and PSD-95 positive area ratios of the Lp082 group significantly increase, and the GFAP positive area ratio significantly decreases. The above results indicate that GOLDGUT-HNU082 can significantly alleviate neuronal damage and further alleviate depression.

### 2.5 GOLDGUT-HNU082 Improving Gut Microbiota Dysbiosis in Depressed Mice

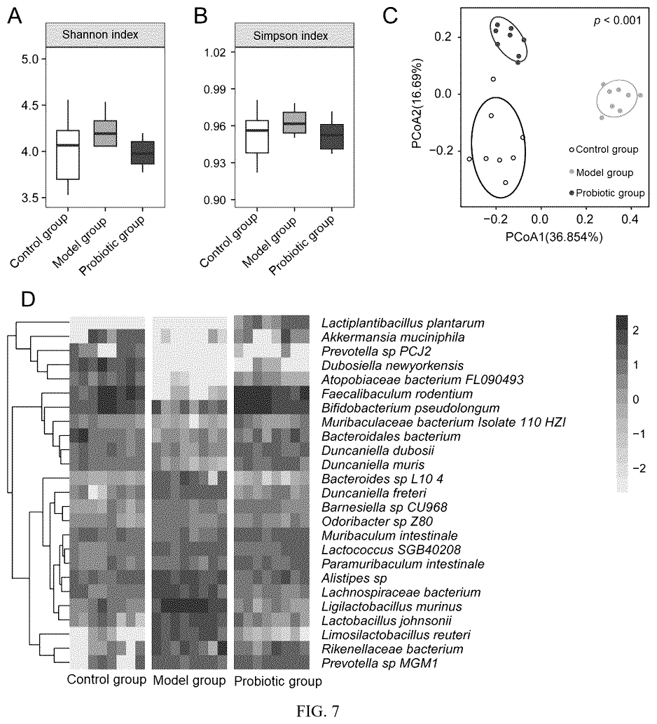

Metagenomic sequencing was performed on the intestinal contents of mice from different groups. The results are as shown in FIG. 7, showing no significant differences in the Shannon indexes and Simpson indexes of the gut microbiota in the mice of groups, which indicates no significant differences in α diversity (A in FIG. 7, B in FIG. 7). Principal coordinate analysis (PCoA) was performed based on Bray-Curtis distance. The results show significant differences in gut microbiota β diversity among the Con group, Mod group, and Lp082 group (C in FIG. 7, p<0.05), indicating significant separation of the gut microbiota structures of the groups. In the above, the gut microbiota structure of the Mod group is away from that of the Con group, while that of the Lp082 group is closer to that of the Con group. The analysis results of differential bacterial species show that compared with the Con group, the Mod group shows a significant increase in the abundance of several harmful bacteria, including Alistipes sp., Lachnospiraceae bacterium, Bacteroides sp. L10_4, Muribaculum intestinale, and Duncaniella freteri; and a significant decrease in the abundance of beneficial bacteria such as Bifidobacterium pseudolongum (D in FIG. 7). After intervention of GOLDGUT-HNU082, the abundance of the above microorganisms is significantly reversed. Additionally, the abundance of another beneficial bacterium, Akkermansia muciniphila, is also increased significantly in the Lp082 group.

The above description of the disclosed embodiments is intended to enable one skilled in the art to implement or use the present disclosure. Various modifications to these embodiments will be readily apparent to one skilled in the art, and the general principles defined herein may be implemented in other embodiments without departing from the spirit or scope of the present disclosure. Therefore, the present disclosure is not limited to the embodiments shown herein but is intended to conform to the widest scope consistent with the principles and novel features disclosed herein.

1. Lactiplantibacillus plantarum GOLDGUT-HNU082, deposited in China General Microbiological Culture Collection Center, with a deposit number of CGMCC NO. 33075 and a deposit date of Dec. 16, 2024, and having a taxonomic name of Lactiplantibacillus plantarum.

2. A method for preparing a drug for alleviating depression, comprising using the Lactiplantibacillus plantarum GOLDGUT-HNU082 according to claim 1 to prepare the drug for alleviating depression.

3. The method according to claim 2, wherein the depression is caused by unpredictable mild stress.
